(19) United States
(12) Patent Application Publication
(10) Pub. No.: US 2023/0084729 A1
(43) Pub. Date: Mar. 16, 2023

(54) REDUCED EMISSIONS USING SYNGAS FERMENTATION

(71) Applicant: CONOCOPHILLIPS COMPANY, Houston, TX (US)

(72) Inventor: Peter N. SLATER, Houston, TX (US)

(73) Assignee: CONOCOPHILLIPS COMPANY, Houston, TX (US)

(21) Appl. No.: 17/879,014

(22) Filed: Aug. 2, 2022

Related U.S. Application Data
(60) Provisional application No. 63/242,268, filed on Sep. 9, 2021.

(51) Int. Cl. C12P 7/08; C07C 1/24; C01B 3/36

(52) U.S. Cl. CPC C12P 7/08; C07C 1/24; C01B 3/36; C01B 2203/025; C01B 2203/06; C01B 2203/062; C01B 2203/1205

(57) ABSTRACT

Methods for reducing or reusing emissions and waste from oil and gas processing facilities are described. Specifically, emission and waste streams can be partially oxidized before being treated in a modified syngas fermentation process with parallel bioreactors to produce commodity chemicals of commercial importance while lowering greenhouse gas emissions. At least one bioreactor is online at all times, offline reactors being emptied to collect product and recharged for use.

### PRIOR RELATED APPLICATIONS

This application claims priority to U.S. Ser. No. 63/242,268, filed Sep. 9, 2021, and incorporated by reference in its entirety for all purposes.

### FEDERALLY SPONSORED RESEARCH STATEMENT

Not applicable.

### REFERENCE TO MICROFICHE APPENDIX

Not applicable.

### FIELD OF THE DISCLOSURE

The disclosure relates generally to processes to reduce the emissions from oil and gas processing facilities, and specifically to the application of a syngas fermentation to ethanol process applied to gas emission streams and other waste streams in the processing facility.

### BACKGROUND OF THE DISCLOSURE

Synthesis gas (hereinafter referred to as “syngas”) is a mixture of hydrogen (H₂) and carbon monoxide (CO), and very often some carbon dioxide (CO₂). Syngas is produced by the gasification of carbonaceous materials, such as coal, petroleum, natural gas, lignite, and even biomass, such as lignocellulosic biomass. It can be produced from virtually any material containing carbon, using many methods such as pyrolysis, tar cracking and char gasification, and steam reformation processes of e.g., methane or natural gas.

Syngas is also a platform intermediate in the chemical and biorefining industries and has a vast number of uses. For example, syngas can be converted into alkanes, olefins, oxygenates, and alcohols. These chemicals can be blended into, or used directly as, diesel fuel, gasoline, and other liquid fuels. Syngas can also be converted into liquid fuels by methanol synthesis, mixed-alcohol synthesis, Fischer-Tropsch process, and syngas fermentation.

Of these uses, the production of ethanol is the most important, as it is used in everything from personal care products and cosmetics to beverages, solvents, and fuel. In fact, ethanol is rapidly becoming a major hydrogen-rich liquid transport fuel around the world. The global ethanol market is estimated to increase at a compound annual growth rate of 1.77% from a market size of 38.826 billion in US dollars (USD) in 2019 to achieve a market size of USD 43.136 billion by the end of 2025. This sharp growth in the global market is attributed to increasing interest in ethanol in Europe, Japan, the USA, and several developing nations.

Syngas fermentation to ethanol is a hybrid thermochemical/biochemical process that takes advantage of the simplicity of the gasification process and the specificity of a microbial fermentation process to deliver ethanol and potentially other chemicals. In more detail, certain microbes ferment combinations of carbon monoxide, hydrogen, and carbon dioxide to produce ethanol with high selectively, according to the following overall reactions:

6CO+3H₂O→C₂H₅OH+4CO₂

6H₂+2CO₂→C₂H₅OH+3H₂O

See also FIG. 1A showing a common pathway for syngas fermentation. The Wood-Ljungdahl pathway is a set of biochemical reactions used by some bacteria and archaea called acetogens and methanogens, respectively. It is also known as the reductive acetyl-coenzyme A (Acetyl-CoA or A-CoA) pathway. This pathway enables these organisms to use hydrogen as an electron donor, and carbon dioxide as an electron acceptor and as a building block for biosynthesis. FIG. 1B shows another pathway, known as the Calvin-Benson-Bassham pathway.

Syngas fermentation systems are known. See FIG. 2A-B for examples of syngas fermentation systems, typically using biomass as a carbon source for the syngas. Though syngas fermentation has been used for many years as an atypical gas-to-liquid process, not all oil and gas processes result in a carbon monoxide- or carbon dioxide-rich stream that can be utilized for the cost-effective, or efficient, ethanol production using such methods. Thus, there exists a need to modify the syngas fermentation process such that it can be more applicable to hydrocarbon production and processing streams in a cost effective and sustainable way. This disclosure provides one or more of those needs.

### SUMMARY OF THE DISCLOSURE

The present disclosure is directed to methods for reducing emissions from oil and gas processing facilities. Specifically, solid or gaseous waste streams normally intended for flaring or stranding are instead partially oxidized to generate a high-carbon monoxide (CO) syngas stream. The CO-rich syngas stream can then be fermented using parallel bioreactors to produce commodity chemicals wherein at least one bioreactor is always running and able to accept syngas stream, while others are offline. Thus, not only are emissions, flaring, and solid waste reduced, but the resulting high-volume commodity chemicals can be monetized, resulting in a more cost-efficient process and/or facility.

In one aspect of the presently disclosed process, a partial oxidizer is used to convert a gaseous carbonaceous stream that would otherwise be flared or stranded into a CO-rich syngas stream. The CO-rich syngas stream can then be fed into an array of two or more bioreactors typically used in syngas fermentation that are operating in parallel in the presently described process. At least one reactor is operating to convert the CO-rich syngas stream to ethanol while at least one reactor is in standby mode, allowing for product isolation and recharging of the bioreactor. This allows for a continuous flow of the CO-rich syngas stream and conversion to ethanol.

For example, when the operating reactor's capacity to convert CO to e.g., ethanol, is expended, the off-line reactor will be switched from standby mode to operating mode, allowing for the first operating reactor to be drained and recharged with media and cells without decreasing or stopping the flow of the CO-rich syngas stream. In some embodiments, the tank can be allowed to settle, or cells otherwise collected, and the top liquor siphoned or drained off, so that recharging needs only media replacement, as the cells largely remain behind. The liquid ethanol can then be separated out from the removed liquid, collected, and sold.

In another aspect of the presently disclosed process, the partial oxidizer can be used to oxidize solid carbon sources to produce a CO-rich syngas stream. For example, solid carbon is often rejected by a methane pyrolysis process. Like the flare gas, this solid carbon can be comminuted as needed and partially oxidized to CO-rich syngas stream before being fed into a syngas fermentation bioreactor. This allows for not only the utilization of solid waste produced from the methane pyrolysis process, but also the generation of commercially needed ethanol while lowering emissions.

In some embodiments, the bioreactor with the expended fermentation material can be physically removed from the parallel set-up and transported to a central facility for ethanol separation and bioreactor recharge process, but it is expected that the fluid itself will be handled on-site or transported a short distance via pipelines.

In some embodiments, at least 2, 3, 4, 5 or 6 bioreactors are in parallel with at least one bioreactor actively converting the CO-rich syngas stream to ethanol at all times. The ethanol can be sold or used as-is, or further processed into other commercial gases, e.g., ethylene.

Any known means for partially oxidizing the gas or solid carbonaceous source can be used in the present processes. However, the most common means will be the use of a reformer or gasifier unit with one or more inlet(s) for the carbonaceous feedstock and a sub-stoichiometric amount of pure oxygen (C+½ O₂→CO), and one or more outlet(s) for the CO-rich syngas stream. Gasifiers convert solids into CO-rich gas streams. Partial oxidizers do the same for gaseous streams. Providing sub-stoichiometric oxygen ensures that the product is CO rather than CO₂.

The present methods include any of the following embodiments in any combination(s) of one or more thereof:

A method of reducing emissions from a hydrocarbon processing facility comprising the steps of: a) obtaining at least one carbon-rich waste emission stream from one or more hydrocarbon or petroleum processes; b) partially oxidizing said carbon-rich waste emission stream in a partial oxidation chamber (POX) to form a carbon monoxide-rich syngas stream; c) introducing said carbon monoxide-rich syngas stream and an optional hydrogen stream into a first bioreactor with a first fermentation fluid comprising at least one microbe in a broth and growing said microbe at conditions to convert said carbon monoxide-rich syngas stream to ethanol in said first bioreactor until said broth is spent; d) introducing said carbon monoxide-rich syngas stream and an optional hydrogen stream into a second bioreactor with a second fermentation fluid comprising with at least one microbe in a broth and growing said microbe at conditions to convert said carbon monoxide-rich syngas stream to ethanol in said second bioreactor until said broth is spent; e) removing said first fermentation fluid from said first bioreactor and isolating said ethanol from said first fermentation fluid simultaneously with step d and recharging said first bioreactor with fresh broth or with fresh broth and fresh cells; f) removing said second fermentation fluid from said second bioreactor and isolating said ethanol from said second fermentation fluid simultaneously with step c and recharging said second bioreactor with fresh broth or with fresh broth and fresh cells; and g) repeating steps c-f one or more times and alternating said first and second bioreactor with each repeat.

A method of reducing emissions from a hydrocarbon processing facility, said method comprising the steps of: a) obtaining at least one carbon-rich waste emission stream from one or more hydrocarbon processes; b) condensing and cooling said waste emission stream to form natural gas liquid (NGL) and a lean gas stream; c) partially oxidizing said lean gas stream in a partial oxidation chamber (POX) to form a CO-rich syngas stream; d) introducing said syngas stream and an optional hydrogen stream into a first bioreactor under pressure with a first fermentation fluid comprising at least one species of microbe in a broth and growing said microbe at conditions to convert said syngas stream to a product in said first bioreactor; e) introducing said syngas stream and an optional hydrogen stream into a second bioreactor with a second fermentation fluid comprising at least one species of microbe in a broth and growing said microbe at conditions to convert said syngas stream to said product in said second bioreactor; f) removing said first fermentation fluid from said first bioreactor and isolating said product from said first fermentation fluid simultaneously with step e and recharging said first bioreactor with fresh broth or with fresh broth and fresh microbe; g) removing said second fermentation fluid from said second bioreactor and isolating said product from said second fermentation fluid simultaneously with step d and recharging said second bioreactor with fresh broth or with fresh broth and fresh microbe; and h) repeating steps d-g one or more times and alternating said first and second bioreactor with each repeat.

A method of producing ethanol, said method comprising the steps of: a) pyrolyzing methane in the presence of a catalyst to split said methane into a hydrogen stream and a solid carbon stream, wherein said pyrolysis does not form a greenhouse gas; b) partially oxidizing said solid carbon stream in a POX to form a carbon monoxide-rich syngas stream; c) introducing said syngas stream and an optional hydrogen stream into a bioreactor unit, wherein said bioreactor unit comprises a plurality of bioreactors in parallel; d) contacting said syngas stream and said optional hydrogen stream with a fermentation fluid comprising microbes and broth in a first subset of said plurality of bioreactors in said bioreactor unit at fermentation conditions; e) converting said syngas stream to ethanol in said first subset of said plurality of bioreactors; f) converting said syngas stream to ethanol in a second subset of said plurality of bioreactors while simultaneous removing said ethanol from said first subset of said plurality of bioreactors and recharging said fermentation fluid; and g) repeating steps c-f and alternating said first and second subsets of bioreactors.

A method of reducing emissions from a hydrocarbon production facility, said method comprising the steps of: obtaining at least one carbon-rich waste emission stream from one or more hydrocarbon production processes; removing one or more hydrocarbon products from said carbon-rich waste emission stream to provide a lean waste stream; partially oxidizing said lean waste stream in a partial oxidation chamber (POX) to form a CO-rich syngas stream using sub-stoichiometric amounts of oxygen so that more CO is produced than CO₂; fermenting said syngas stream and an optional hydrogen stream in parallel bioreactors to produce a bioproduct, such that a first bioreactor is online and fermenting while a second bioreactor is offline for collection of said bioproduct and replenishing of said second bioreactor, and alternating said first and second bioreactor with each cycle. The waste stream may be first comminuted if solid and not yet in powder form. Removing hydrocarbon products to make a lean waste stream can include any method known in the art, e.g., fractionation, chilling, evaporation, precipitation, extraction, combinations thereof, and the like.

Any method herein described, wherein all steps are performed at a same site. Alternatively, said removing step and recharging steps e-f are performed off-site from steps a-d.

Any method herein described, said first bioreactor and said second bioreactor are sequentially moved off-site and said removing step and recharging steps e-f are performed off-site and then first bioreactor and said second bioreactors are sequentially returned on-site.

Any method herein described, wherein said first and second bioreactors are semi-batch bioreactors.

Any method herein described, further comprising the step of dehydrating said ethanol to form ethylene.

Any method herein described, wherein said carbon-rich emission stream is stranded gas, flaring gas or both. Alternatively, said carbon-rich emission stream is solid carbon from a methane pyrolysis process.

Any method herein described, wherein said at least one carbon-rich waste emission stream is cooled and natural gas liquids are condensed therefrom and stored or sold, and a remaining lean gas is sent to said POX. Alternatively, said at least one carbon-rich waste emission stream is a solids stream and said solid is comminuted and sent to said POX.

Any method herein described, wherein microbes are collected from said fermentation fluid and used for recharging said first and second bioreactors and product is isolated from a remaining fluid.

Any method herein described, wherein microbes in said fermentation fluid are lysed and product is isolated from said broth and said lysed microbes.

In some embodiments of the present disclosure, the bioreactor system may comprise a cell amplification tank or bioreactor in which the microbes are initially cultured and where growth conditions may vary somewhat from optimal production conditions. For example, it is common for bacterial products to be produced by first culturing a bacteria in a growth medium aerobically (e.g., about 40% dissolved oxygen (DO)) until sufficient cell mass is obtained, e.g., an Optical Density (OD) of >2, >3, >4, >5 or >6 is reached; further culturing said bacteria under oxygen lean conditions (e.g., <5% DO) and sparging the head space with air or O₂ containing gas until product is formed; and isolating said product from said bacteria, said growth medium or both. See e.g., U.S. Ser. No. 10/920,251. Likewise, anaerobic microbes may also be cultured for cell growth under one set of conditions, and product formation optimized to another set of conditions.

While certain embodiments of the disclosure are directed to the production of ethanol by anaerobic fermentation using CO and optionally H₂ as the primary substrate, it should be appreciated that the presently described methods are applicable to production of alternative saleable products such as ethylene, acetate, butyrate, propionate, caproate, propanol, and butanol, and hydrogen.

Most frequently, syngas fermentations use acetogenic anaerobes, such as the genus Moorella, Clostridia, Ruminococcus, Acetobacterium, Eubacterium, Butyribacterium, Oxobacter, Methanosarcina, Methanosarcina, and Desulfotomaculum. In addition, several mycobacterial strains, such as Mycobacterium flavescens, Mycobacterium gastri, Mycobacterium neoaurum, Mycobacterium parafortuitum, Mycobacterium peregrinum, Mycobacterium phlei, Mycobacterium smegmatis, Mycobacterium tuberculosis, and Mycobacterium vaccae, can also grow on carbon monoxide (CO) as the sole source of carbon and energy.

Specific strains suitable for use in the presently disclosed methods include those of strains of Clostridium ljungdahlii, including those described in WO2000068407, U.S. Pat. Nos. 5,173,429, 5,593,886, 6,368,819, WO1998000558 and WO2002008438, Clostridium carboxydivorans (Liou, 2009) and Clostridium autoethanogenum (Abrini, 1994). Suitable Moorella include Moorella sp HUC22-1, (Sakai, 2004), M. thermoacetica, and M. thermoautotrophica. Other species include those of the genus Carboxydothermus (Svetlichny, 1991), Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa, 2006).

One exemplary microbe suitable for use in the present method is Clostridium autoethanogenum having the identifying characteristics of the strain deposited as Deposit Number 19630, on Oct. 19, 2007, at the German Resource Centre for Biological Material (DSMZ), located at Inhoffenstrape 7B, Braunschweig, Germany, D-38124. Another embodiment uses DSMZ 10061. Examples are provided in WO2007117157, WO2008115080, WO2009022925, US20100317074, US20130217096, WO2009064201, U.S. Pat. No. 8,178,330 and US20110144393 all of which are incorporated herein by reference for all purposes.

In addition to anaerobic bacteria, aerobic bacteria and/or yeast can be genetically modified to grow on one-carbon precursors such as CO. The Wood-Ljungdahl pathway (FIG. 1A) allows acetogenic bacteria to grow on a number of one-carbon substrates, such as carbon dioxide, formate, methyl groups, or CO. This pathway may be used to convert microbes into useful microbes herein.

For example, utilitarian CO oxidation which is coupled to the generation of energy for growth is achieved by aerobic and anaerobic eu- and archaebacteria. They belong to the physiological groups of aerobic carboxidotrophic, facultatively anaerobic phototrophic, and anaerobic acetogenic, methanogenic or sulfate-reducing bacteria. The key enzyme in CO oxidation is CO dehydrogenase which is a molybdo iron-sulfur flavoprotein in aerobic CO oxidizing bacteria and a nickel-containing iron-sulfur protein in anaerobic ones. In carboxydotrophic and phototrophic bacteria, the CO-born CO₂ is fixed by ribulose bisphosphate carboxylase in the reductive pentose phosphate cycle. In acetogenic, methanogenic, and probably in sulfate-reducing bacteria, carbon monoxide dehydrogenase/acetyl-CoA (CODH/acetyl-CoA) synthase directly incorporates CO into acetyl-CoA. Thus, these enzymes can be inserted into other bacteria or yeast thereby allowing them to ferment syngas.

Recently, WPS-2 and AD3 bacteria were discovered in Antarctica that can scavenge hydrogen, carbon monoxide and carbon dioxide from the air to stay alive. These bacteria may also be suitable for use herein. Analysis of the large subunit of type I ribulose-1,5-biphosphate carboxylase/oxygenase genes (rbcL) revealed RuBisCO types similar to proteobacteria and actinobacteria, suggesting that diverse bacteria are capable of assimilating carbon dioxide through the Calvin-Benson-Bassham cycle (FIG. 1B). Thus, the genus of microbes suitable for use herein is quite large, including those microbes that can naturally grow on syngas and those that can be genetically modified to do so.

The term “broth” or “media” are used interchangeable herein to refer to a liquid material that contains vitamins and minerals sufficient to permit growth of the microbe.

The term “fermentation fluid” refers to the broth plus microbes collectively.

The term “syngas” refers to a gas mixture that contains at least a portion of carbon monoxide, hydrogen, and/or carbon dioxide produced by gasification and/or reformation of a carbonaceous feedstock.

“Microbes” herein are single cell organisms such as aerobic and anaerobic bacteria, archaebacteria, yeast and algae. Even when written in the singular, microbe is never singular but always includes a large population of cells, except that a species of microbe refers to a single species.

The term “bioreactor” refers to a fermentation device consisting of one or more vessels, bioreactors and/or towers or piping arrangements where the fermentation occurs, which includes the continuous stirred tank reactor (CSTR), an immobilized cell reactor, a gas-lift reactor, a bubble column reactor (BCR), a membrane reactor, such as a Hollow Fiber Membrane Bioreactor (HFMBR), a trickle bed reactor (TBR), monolith bioreactor, forced or pumped loop bioreactors, semi-batched bio-reactors, or combinations thereof, or other vessel or device suitable for gas-liquid contact and growth of microbes.

Unless the context requires otherwise, the phrases “fermenting”, “fermentation process” or “fermentation reaction” and the like, as used herein, are intended to encompass both the growth phase and product biosynthesis phase of the process, unless the phases are clearly being discussed separately.

The use of the word “a” or “an” when used in conjunction with the term “comprising” in the claims or the specification means one or more than one unless the context dictates otherwise. The use of the term “or” in the claims is used to mean “and/or” unless explicitly indicated to refer to alternatives only or if the alternatives are mutually exclusive.

The term “about” means the stated value plus or minus the margin of error of measurement or plus or minus 10% if no method of measurement is indicated.

The terms “comprise”, “have”, “include” and “contain” (and their variants) are open-ended linking verbs and allow the addition of other elements when used in a claim. The phrase “consisting of” is closed and excludes all additional elements. The phrase “consisting essentially of” excludes additional material elements but allows the inclusion of non-material elements that do not substantially change the nature of the invention, such as buffers, vitamins, aeration methods, and the like.

Any claim or claim element introduced with the open transition term “comprising,” may also be narrowed to use the phrases “consisting essentially of” or “consisting of,” and vice versa. However, the entirety of claim language is not repeated verbatim in the interest of brevity herein.

The following abbreviations are used herein:

| ABBREVIATION | TERM |
|---|---|
| BCR | Bubble Column Reactor |
| CAGR | Compound Annual Growth Rate |
| CO | Carbon monoxide |
| CO₂ | Carbon dioxide |
| CSTR | Continuous Stirred Tank Reactor |
| DO | Dissolved oxygen |
| EtOH | Ethanol |
| GHG | Greenhouse gas |
| HFMBR | Hollow Fiber Membrane Bioreactor |
| IPA | Isopropyl alcohol |
| MRU | Mechanical refrigeration unit |
| NGL | Natural Gas Liquid |
| OD | Optical density |
| POX | Partial oxidation unit |
| Syngas | Synthesis gas |
| TBR | Trickle Bed Reactor |

### BRIEF DESCRIPTION OF THE DRAWINGS

FIG. 1A. The Wood-Ljungdahl pathway is a set of biochemical reactions used by some bacteria and archaea called acetoge ns and methanogens, respectively. Also known as the reductive acetyl-coenzyme A (Acetyl-CoA) pathway, this pathway enables these organisms to use hydrogen as an electron donor, and carbon dioxide as an electron acceptor and as a building block for biosynthesis. In this pathway carbon dioxide is reduced to carbon monoxide and formic acid or directly into a formyl group, the formyl group is reduced to a methyl group and then combined with the carbon monoxide and Coenzyme A to produce acetyl-CoA. Two specific enzymes participate on the carbon monoxide side of the pathway: CO dehydrogenase and acetyl-CoA synthase. The former catalyzes the reduction of the CO₂ and the latter combines the resulting CO with a methyl group to give acetyl-CoA.

FIG. 1B The Calvin-Benson-Bassham cycle in R. eutropha. Ribulose-5-phosphate is phosphorylated by the enzyme phosphoribulose kinase (CbbP). The resulting compound, ribulose-1,5-bisphosphate is then carboxylated by ribulose-1,5-bisphosphate carboxylase/oxygenase (RuBisCO) (CbbL and CbbS). The outcome of this carboxylation are two molecules of 3-phosphoglycerate (3-PGA). 3-PGA is phosphorylated by phosphoglycerate kinase (CbbK) to yield 1,3-bisphosphoglycerate (1,3-BP). 1,3-bisphosphoglycerate is reduced by NADPH to yield NADP⁺ and glyceraldehyde-3-phosphate (GAP) by glyceraldehyde-3-phosphate dehydrogenase (CbbG). GAP is then converted fructose-6-phosphate (F6P) by aldolase (CbbA) and fructose bisphosphatase (CbbF). The reversible reactions of the reductive pentose phosphate cycle involving erythrose-4-phosphate, fructose-6P, sedoheptulose-7P, xylulose-5P, and ribose-5-P are catalyzed by the enzymes: transketolase (CbbT), fructose-bisphosphate aldolase (CbbA), fructose/sedoheptulose bisphosphatase (CbbF), ribulose-5-epimerase (CbbE), and triosephosphate isomerase (TpiA). Ribose-5P is isomerized by ribose-5-phosphate isomerase (RpiA) to yield ribulose-5P, which can then be put back into the cycle.

FIG. 2A. Prior art: Syngas fermentation system.

FIG. 2B. Prior art: Alternate syngas fermentation system.

FIG. 3. Preparation of gaseous products for use in bioreactor.

FIG. 4. Cell amplification for use in bioreactor.

FIG. 5. Parallel bioreactors for syngas fermentation.

FIG. 6. Cell and broth treatment to produce product, such as ethanol.

FIG. 7. Cell separator and broth treatment to produce product, such as ethanol.

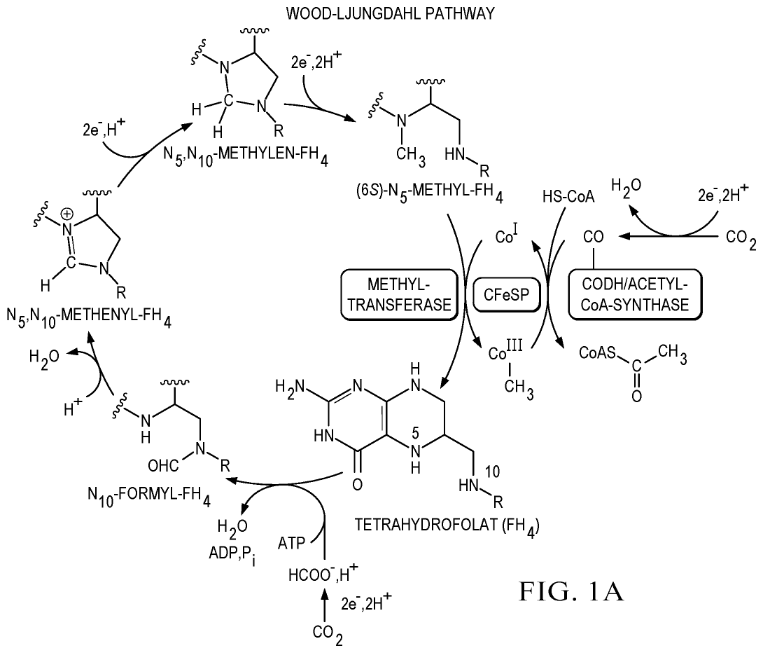

### DESCRIPTION OF EMBODIMENTS OF THE DISCLOSURE

The present disclosure provides a novel method of reducing emissions and waste in oil-and-gas processing sites by partially oxidizing carbon-rich emission and waste streams into syngas before fermenting the syngas in parallel bioreactors to commodity chemicals, such as ethanol.

In more detail, carbon-rich emission and waste streams such flare gas, stranded gas, or solid carbon-rich byproduct are collected and partially oxidized in a reformer or gasifier unit. The partial oxidization transforms the carbon-rich emission and waste streams into a CO-rich syngas stream. In many cases, it may be beneficial to collect useful gases or other materials, such as Natural Gas Liquids (NLG), before the oxidation step, as these products have independent sale value.

Carbon-rich solid waste streams can also be partially oxidized into syngas. For example, methane pyrolysis with thermo-catalysis is used to extract the carbon in natural gas in a solid form rather than emitting in a gaseous form. While this method reduces the emission from a typical extraction process and reduces greenhouse gases (GHG), the resulting solid carbon waste will still need to be addressed. Using the presently described methods, this solid carbon waste can also be converted to ethanol, thus further reducing facility waste.

The CO-rich syngas stream will typically contain a major proportion of CO, such as at least about 15%-75% CO, 20%-65% CO, 20%-60% CO, or 20%-55% CO by volume. However, lower or higher concentrations of CO can also be used in the present methods. The CO-rich syngas stream may also contain some CO₂ for example, about 1%-85%, or 5%-30% or 10-25% CO₂ by volume.

In some embodiments, an optional hydrogen stream can be fed alongside the CO-rich syngas stream. The presence of hydrogen may result in an improved overall efficiency of ethanol production, but its need will depend on the microbe used for fermentation, as well as the product and the gas content of the syngas stream.

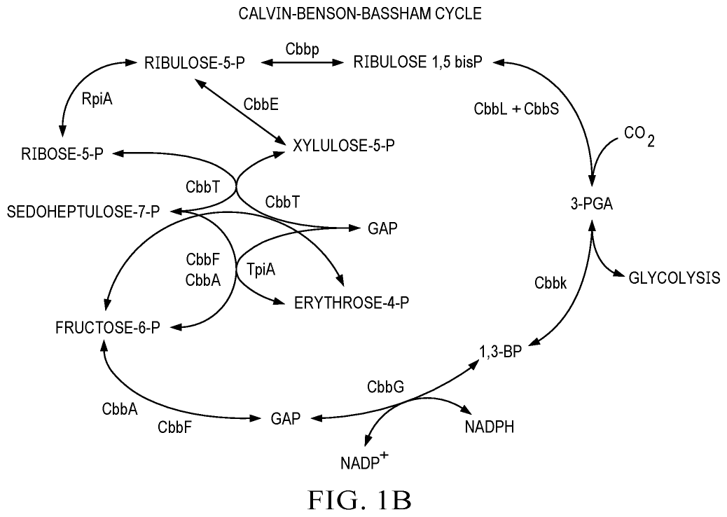

In contrast to the typical syngas bioreactor setups, such as shown in FIG. 2A-B, the present system has an array of at least two bioreactors operating in parallel. This allows for the bioreactors to be in alternating operating and standby mode. That is, a first subset of bioreactors can be in operating mode and thus receiving and fermenting the CO-rich syngas stream to produce alcohol, while a second subset of bioreactors is in standby mode being recharged. Once the fermentation material in the first subset of bioreactors becomes spent and is no longer able to convert the syngas to a product, the second subset of reactors can be changed to operating mode by diverting input streams thereto, allowing them to convert the CO-rich syngas to a commercial product. Thus, the system can be operated without stopping and/or slowing the conversion of the syngas and thus avoid contributing any waste gases to the environment.

In use, the fermenters are monitored to control cell growth, growth conditions, and product levels. Thus, the bioreactor is typically equipped with a variety of sensors to measure various conditions such as pH, temperature, O₂ content, CO and/or CO₂ content, H₂ content, turbidity or OD, concentrations of nutrients, pressure, and products like acetic acid and ethanol, and the like. Since CO is sparingly soluble, the fermenter is typically run under pressure.

In some embodiments, the subset of bioreactors that has spent material can be physically removed from the bioreactor unit and transported to a central facility for the removal of products and/or regeneration steps, without affecting the other subset of reactors that are in operating mode. This may be a practical means of handling flare gas in the field, e.g., the Bakken reservoir, at least for proof-of-concept stage. However, ideally, it will probably be more cost effective to put most functions on site or within piping distance of the gas source.

By utilizing this modified syngas fermentation process, oil-and-gas production or processing sites can significantly reduce their emissions, both in the form of flaring or GHG release, as well as their solid waste from certain processes. Further, because commercially important commodity chemicals, such as ethanol, are generated, the facilities can use these generated feedstocks in other processes on site or sell them, thus improving the cost-efficiency of their processes, facility, and/or site.

Many products can be produced in a syngas fermenter, including ethanol and acetic acid, which derive directly from acetyl-CoA without the expense of ATP. Ethanol, which can be recovered by distillation, is the most prominent product. Acetic acid requires more elaborate recovery, such as extraction, but is a high-volume chemical and could potentially be produced by oxidation of ethanol. Ethylene, globally one of the highest selling gases, can be formed by dehydration of the ethanol.

An additional ATP is expended by the cells to condense two acetyl-CoA to butyryl-CoA, which is converted to butyric acid and then to butanol in steps similar to ethanol production. Propionic acid, propanol, hexanoic acid, hexanol, acetone, iso butanol, butanediol, amino and fatty acids are other potential products proposed from syngas fermentation. Although less common, the accumulation of butyric acid, hexanoic acid, butanol and hexanol has been demonstrated for C. carboxidivorans. In addition, a biological water-gas shift is proposed to produce H₂, and syngas can be biologically converted to methane so that syngas energy and subsequent products might be obtained from biological conversion to natural gas.

Culturing of the microbes used in the methods of the present disclosure may be conducted using any number of processes known in the art for culturing and fermenting substrates using microbes. By way of example, those processes generally described in the following articles using gaseous substrates for fermentation, may be utilized: (i) Klasson, 1991; (ii) Klasson, 1991b; (iii) Klasson, 1992; (iv) Vega, 1989; (vi) Vega, 1989; (vii) Vega, 1990; all of which are incorporated herein in their entirety by reference for all purposes.

Additionally, any known fermentation conditions can be utilized as the optimum reaction conditions will depend partly on the type of microbe used. However, in general, it is preferred that the fermentation be performed at pressures higher than ambient pressure and under anaerobic conditions for certain microbes. Operating at increased pressures allows a significant increase in the rate of CO transfer from the gas phase to the liquid phase where it can be taken up by the microbe as a carbon source to produce ethanol. This in turn means that the retention time (defined as the liquid volume in the bioreactor divided by the input gas flow rate) can be reduced when bioreactors are maintained at elevated pressure rather than atmospheric pressure.

The presently disclosed methods are described below for the formation of ethanol from flare gas. However, this is exemplary only, and the invention can be broadly applied to other carbon rich waste sources on hydrocarbon processing sites and to the production of other products. The following embodiments are intended to be illustrative only, and not unduly limit the scope of the appended claims.

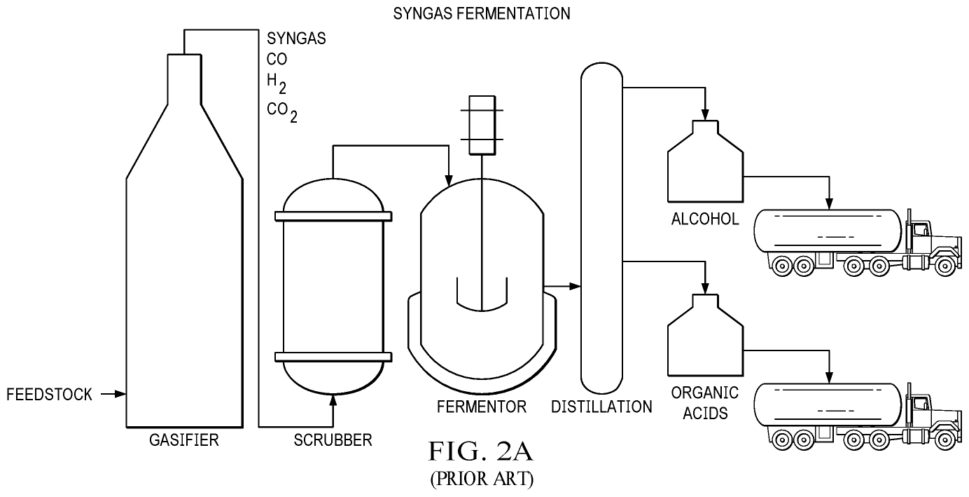

FIG. 3 illustrates one embodiment of the process, wherein the carbon-rich flare gases are fed using flare gas line 301 into a mechanical refrigeration unit (MRU) 303. At this stage, the incoming flare gas is cooled and condensed to capture any Natural Gas Liquids (NGL). These NGLs are transported via NGL line 313 to a storage container 315 for storing or direct sales of the NGL.

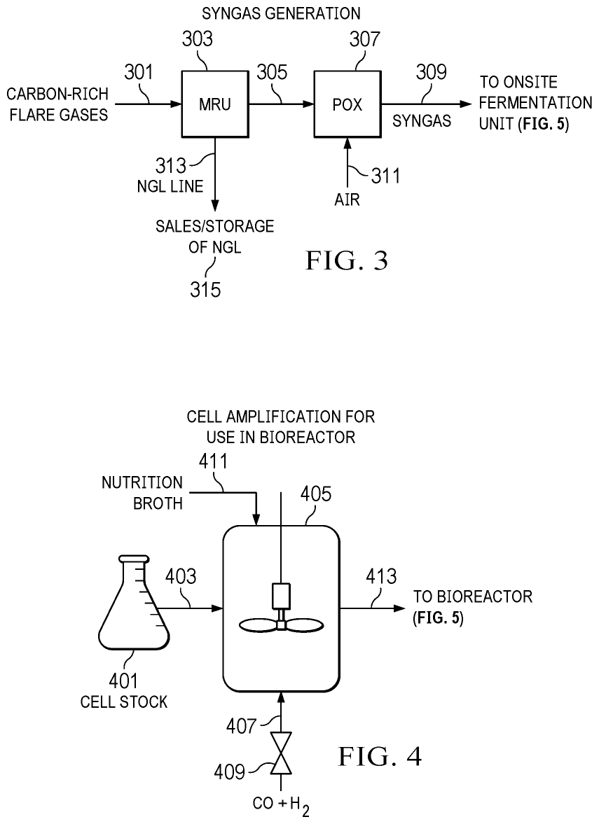

The gas left after the condensing of natural gas, also known as lean gas, is passed from the MRU using a lean gas line 305 to a catalytic partial oxidation unit (POX) 307. At POX, the lean gas is mixed with air fed in from air intake line 311 to catalytically oxidize carbon-rich lean gas into CO-rich gas stream along with H₂. If desired, additional H₂ can be fed into the system at 307 or a separate line added for same. Steam reforming at this stage may also produce N₂ and some residual CH₄. Line 309 carries the syngas to the bioreactors below, described in FIG. 5. Further, although not detailed herein it is preferred that the catalysts are regenerated, for example as described in U.S. Pat. No. 7,524,786.

Syngas catalysts can be any known in the art, including e.g., Group VIII noble metals, alkali metals, metal oxide systems, zeolite, silica, and alumina supported metal catalysts, zeolite-iron material or cobalt-molybdenum carbide materials, and the like.

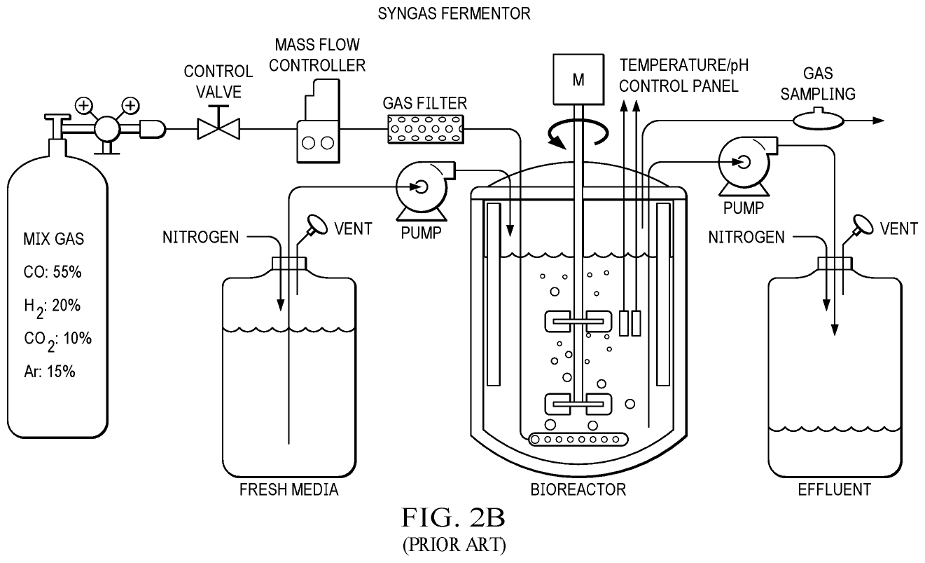

FIG. 4 shows an optional exemplary set-up to amplify cells from cell stocks for inoculating the bioreactors. Flask 401 contains cells, which may be liquid cells, dried cells or frozen cells, and are used to inoculate container 405, via cell line 403 or manually. Gas intake line 407 feeds the CO and H₂ via opening valve 409 and line 411 feeds in broth. Once sufficiently multiplied, e.g., to stationary phase, anaerobic cells are then fed through cell transfer line 413 to the bioreactors in FIG. 5.

In some embodiments, the cells may be collected by filtration or centrifugation to reduce the inoculation volume, but minimal handling is preferred. As before, the inoculation of the larger bioreactors could be manual, but in general a closed system is preferred to maintain sterility and anaerobic conditions. Further, it is preferred that sensors are included (not shown) so that the cell amplifier unit may be run more or less continuously, supplying broth and removing cells for use as needed.

FIG. 5 describes the onsite fermentation unit consisting of parallel bioreactors 501 and 502. The syngas produced at POX is transferred via syngas line 309 to the bioreactors. Pumps and compressors are omitted for clarity but are placed as needed to move liquids and maintain a higher pressure in the bioreactor to encourage CO dissolution into the broth. Also not shown is the mixer and/or bubbler inside the bioreactors that serves to keep cells and fluid moving, but preferably, the gas feeds in at the bottom and thus bubbles up from the bottom (as shown in FIG. 2B) to aid the mixer and gas solubility. In addition, this system has been simplified for clarity, but sensors will be included as we well as additional lines, as needed to control pH, sample fermentation fluid, and the like.

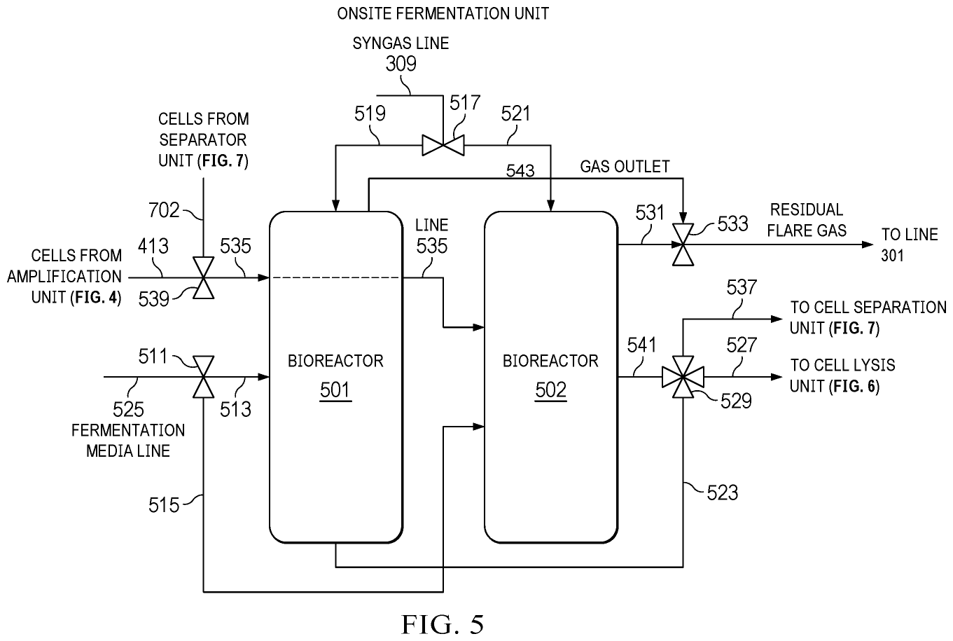

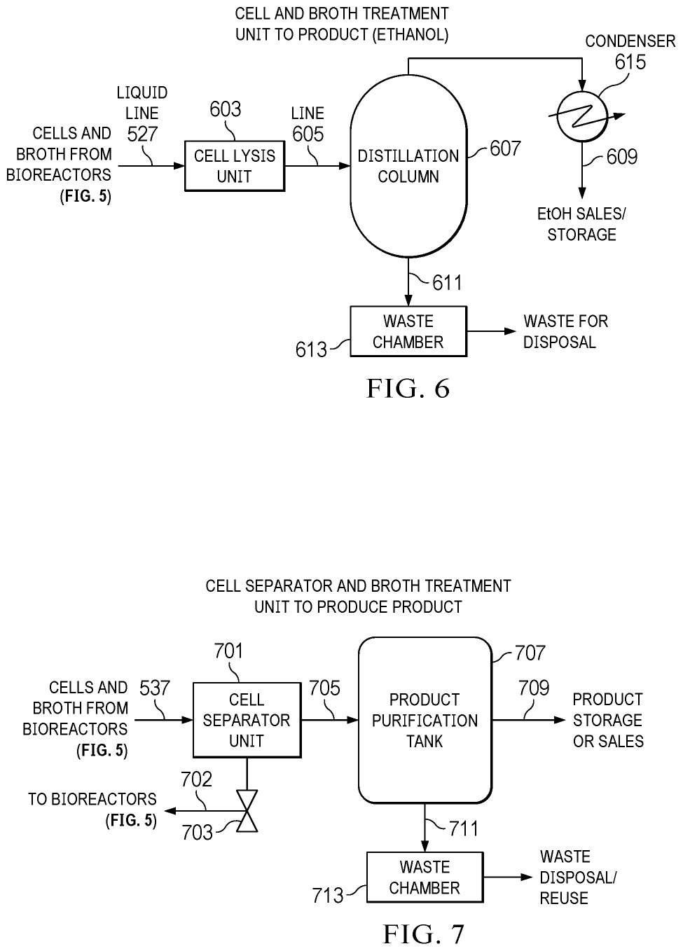

In the two bioreactor system of FIG. 5, fermentation first occurs in bioreactor 501 while bioreactor 502 is on standby. Fermentation broth comes in from media line 525 and is fed into bioreactor 501 via 513 controlled by valve 511. Syngas from gas line 309 is transferred into bioreactor 501 by line 519 controlled by valve 517. Cells from cell line 413 from cell amplification unit (FIG. 4) or line 702 from cell separator unit (FIG. 7) are transferred into the bioreactor 501 via line 535 controlled by valve 539. After the completion of fermentation process, fluids from bioreactor 501 can be transferred to the separation unit described in FIG. 7 via line 523 and then line 527 controlled by 4-way valve 529. The spent cells and broth mixture can also be transferred to the cell lysis unit in FIG. 6 via line 537 also controlled by valve 529.

After the completion of fermentation of syngas in bioreactor 501, for example, when the broth or the cells are exhausted, valve 511 is switched to feed in broth via lines 525 to bioreactor 502 via line 515. Line 535 can be re-routed to feed in cells into bioreactor 502. Valve 517 sends syngas via lines 309, 521 to bioreactor 502 and the fer[mentation process repeats. Excess gas from bioreactor 501 is removed by gas outlet line 543 and from bioreactor 502 via line 531 controlled by valve 533 and can either be flared or connected back to gas input line 301 or otherwise handled. Once product maximum is reached in bioreactor 502, the broth and cells are transferred via line 541 to either cell lysis unit in FIG. 6 or cell separation unit in FIG. 7 controlled by the 4-way valve 529.

Liquid from the various fermentations is collected in the cell lysis unit 603 where the cells are lysed. Any number of methods including heat, sonication, alkali, acid, enzymes, combinations thereof, and the like can be used for cell lysis. Lines for ingredients, such as lysis buffer, are added as needed but not shown herein for clarity. The lysed cell solution is fed through line 605 into a distillation column 607 where the cell debris and broth residue are separated from ethanol. The distilled ethanol is cooled at the condenser 615 transported through ethanol line 609 for storage and sales. The spent broth media with cell debris is passed through line 611 and collected in waste chamber 613 for proper disposal and/or other uses.

An alternative embodiment is shown in FIG. 7 where the fermentation fluid is instead sent to a cell separator unit 701 which separates the cells from the broth by e.g., filtration, settling, centrifugation, liquid-liquid extraction, perstraction, pervaporation, gas stripping, and the like. The collected cells plus residual fluids are sent back via line 702 controlled by valve 703 to the bioreactors for recharging whichever unit is offline. The broth minus cells is passed through line 705 into e.g., a product purification tank or column 707 where product is separated from broth in any known manner. Additional units are added as needed, for example a catalytic dehydration unit with aluminum oxide catalyst may be added to convert ethanol to ethylene (not shown). The product is transported through line 709 for storage and/or sales. The spent broth media is passed through line 711 and collected in waste chamber 713 for proper disposal and/or reuse.

Whether one collects product from the broth and the cells or just the broth depends in large part on the product, as some products are efficiently secreted/excreted into the media, and others are also found inside the cell, in which case the cells need to be lysed to obtain significant product.

The process is not expected to change much even with solid materials, since everything will already be gaseous at the inlet to the fermenters. However, the solids will probably be first comminuted and or ground as needed, and they may contain aromatics and polyaromatics that are worth recovering and specific recovery unit(s) added for same. Thus, upstream grinding and recovery units will be added to the process described herein.

The following references are incorporated by reference in their entirety for all purposes.

U.S. Pat. No. 5,173,429 Clostridium ljungdahlii, an anaerobic ethanol and acetate producing microorganism

U.S. Pat. No. 5,593,886 Clostridium strain which produces acetic acid from waste gases

U.S. Pat. No. 6,225,469 Biological production of acetic acid from waste gases

U.S. Pat. No. 6,368,819 Microbial process for the preparation of acetic acid as well as solvent for its extraction from the fermentation broth

U.S. Pat. No. 7,285,402 Methods for increasing the production of ethanol from microbial fermentation

U.S. Pat. No. 7,524,786 Regeneration of synthesis gas catalysts

U.S. Pat. No. 7,972,824 WO2007117157 Microbial fermentation of gaseous substrates to produce alcohols

U.S. Pat. No. 8,119,378 Microbial alcohol production process

U.S. Pat. No. 8,178,330B2 Fermentation of gaseous substrates

U.S. Pat. Nos. 8,222,013, 8,852,918 US20130217096 Novel bacteria and methods of use thereof

U.S. Pat. No. 8,293,509 WO2008115080 Alcohol production process

U.S. Pat. Nos. 8,376,736, 8,383,376, 9,127,296, 9,890,399, U.S. Ser. No. 10/435,722, US2020002734 US2020048665 US20100317074 Improved carbon capture in fermentation

U.S. Pat. No. 8,658,408 US2011144393 Production of butanediol by anaerobic microbial fermentation

WO2000068407 Clostridium strains which produce ethanol from substrate-containing gases

WO2009022925 Processes of producing alcohols

WO2009064201 Use of carriers in microbial fermentation

U.S. Pat. No. 5,173,429 Clostridium ljungdahlii, an anaerobic ethanol and acetate producing microorganism

U.S. Pat. No. 6,368,819 Microbial process for the preparation of acetic acid as well as solvent for its extraction from the fermentation broth

U.S. Pat. No. 5,807,722 Biological production of acetic acid from waste gases with Clostridium ljungdahlii

WO2009058028 Improved carbon capture in fermentation

US20100311104 Novel bacteria and methods of use thereof

US2011059499 Microbial alcohol production process

US20110144393 Production of butanediol by anaerobic microbial fermentation

Abrini, et al, Abrini, et al, Archives of Microbiology 161: 345-351 (1994).

Klasson, et al., Bioconversion of synthesis gas into liquid or gaseous fuels. Enzyme and Microbial Technology. 14: 602-608 (1992).

Klasson, et al., Bioreactors for synthesis gas fermentations resources. Conservation and Recycling, 5: 145-165 (1991).

Klasson, et al., Bioreactor design for synthesis gas fermentations. Fuel. 70: 605-614 (1991b).

Liou, et al., Clostridium carboxidivorans sp. nov., a solvent-producing clostridium isolated from an agricultural settling lagoon, and reclassification of the acetogen Clostridium scatologenes strain SL1 as Clostridium drakei sp. nov. International Journal of Systematic and Evolutionary Microbiology 55(5): 2085-2091 (2005).

Sakai, et al, Ethanol production from H₂ and CO₂ by a newly isolated thermophilic bacterium, Moorella sp. HUC22-1, Biotechnology Letters 26(20): 1607-1612 (2004).

Simpa, et. al., Microbial CO conversions with applications in synthesis gas purification and bio-desulfurization, Critical Reviews in Biotechnology, 26: 41-65 (2006).

Svetlichny, et al., Carboxydothermus hydrogenoformans gen. nov., sp. nov., a CO-utilizing thermophilic anaerobic bacterium from hydrothermal environments of Kunashir Island, Systematic and Applied Microbiology 14: 254-260 (1991).

Vega, et al., Study of gaseous substrate fermentations: Carbon monoxide conversion to acetate. 1. Batch culture. Biotechnology and Bioengineering. 34(6): 774-784 (1989).

Vega, et al., Study of gaseous substrate fermentation: Carbon monoxide conversion to acetate. 2. Continuous culture. Biotechnology and Bioengineering. 34(6): 785-793 (1989).

Vega, et al., Design of bioreactors for coal synthesis gas fermentations. Resources, Conservation and Recycling 3: 149-160 (1990).

1. A method of reducing emissions from a hydrocarbon processing facility comprising the steps of:
a) obtaining at least one carbon-rich waste emission stream from one or more hydrocarbon processes;
b) partially oxidizing said carbon-rich waste emission stream in a partial oxidation chamber (POX) to form a carbon monoxide-rich syngas stream;
c) introducing said carbon monoxide-rich syngas stream and an optional hydrogen stream into a first bioreactor with a first fermentation fluid comprising at least one microbe in a broth and growing said microbe at conditions to convert said carbon monoxide-rich syngas stream to ethanol in said first bioreactor until said broth is spent;
d) introducing said carbon monoxide-rich syngas stream and an optional hydrogen stream into a second bioreactor with a second fermentation fluid comprising at least one microbe in a broth and growing said microbe at conditions to convert said carbon monoxide-rich syngas stream to ethanol in said second bioreactor until said broth is spent;
e) removing said first fermentation fluid from said first bioreactor and isolating said ethanol from said first fermentation fluid simultaneously with step d and recharging said first bioreactor with fresh broth or with fresh broth and fresh cells;
f) removing said second fermentation fluid from said second bioreactor and isolating said ethanol from said second fermentation fluid simultaneously with step c and recharging said second bioreactor with fresh broth or with fresh broth and fresh cells; and
g) repeating steps c-f one or more times and alternating said first and second bioreactor with each repeat.

2. The method of claim 1, wherein all steps a-g are performed at a same site.

3. The method of claim 1, wherein said removing step and recharging steps e-f are performed off-site from steps a-d.

4. The method of claim 1, said first bioreactor and said second bioreactor are sequentially moved off-site and said removing step and recharging steps e-f are performed off-site and then first bioreactor and said second bioreactors are sequentially returned on-site.

5. The method of claim 1, wherein said first and second bioreactors are semi-batch bioreactors.

6. The method of claim 1, further comprising the step of dehydrating said ethanol to form ethylene.

7. The method of claim 1, wherein said carbon-rich emission stream is stranded gas, flaring gas or both.

8. The method of claim 1, wherein said carbon-rich emission stream is solid carbon from a methane pyrolysis process.

9. The method of claim 1, wherein said microbe is selected from the Moorella, Clostridia, Ruminococcus, Acetobacterium, Eubacterium, Butyribacterium, Oxobacter, Methanosarcina, Methanosarcina, and Desulfotomaculum genus.

10. The method of claim 1, wherein said microbe is Clostridium carboxydivorans, Clostridium ljungdahlii, Clostridium autoethanogenum, Morella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, or Desulfotomaculum kuznetsovii.

11. The method of claim 1, wherein said at least one carbon-rich waste emission stream is cooled and natural gas liquids are condensed therefrom and stored or sold, and a remaining lean gas is sent to said POX.

12. The method of claim 1, wherein said at least one carbon-rich waste emission stream is a solids stream and said solid is comminuted and sent to said POX.

13. A method of reducing emissions from a hydrocarbon processing facility, said method comprising the steps of:
a) obtaining at least one carbon-rich waste emission stream from one or more hydrocarbon processes;
b) condensing and cooling said waste emission stream to form natural gas liquid (NGL) and a lean gas stream;
c) partially oxidizing said lean gas stream in a partial oxidation chamber (POX) to form a CO-rich syngas stream;
d) introducing said syngas stream and an optional hydrogen stream into a first bioreactor under pressure with a first fermentation fluid comprising at least one species of microbe in a broth and growing said microbe at conditions to convert said syngas stream to a product in said first bioreactor;
e) introducing said syngas stream and an optional hydrogen stream into a second bioreactor under pressure with a second fermentation fluid comprising at least one species of microbe in a broth and growing said microbe at conditions to convert said syngas stream to said product in said second bioreactor;
f) removing said first fermentation fluid from said first bioreactor and isolating said product from said first fermentation fluid simultaneously with step e and recharging said first bioreactor with fresh broth or with fresh broth and fresh microbe;
g) removing said second fermentation fluid from said second bioreactor and isolating said product from said second fermentation fluid simultaneously with step d and recharging said second bioreactor with fresh broth or with fresh broth and fresh microbe; and
h) repeating steps d-g one or more times and alternating said first and second bioreactor with each repeat.

14. The method of claim 13, where said waste emission stream comprises a comminuted solid waste.

15. The method of claim 13, where said waste emission stream comprises a gaseous waste.

16. The method of claim 13, wherein microbes are collected from said fermentation fluid and used for recharging said first and second bioreactors and product is isolated from a remaining fluid.

17. The method of claim 13, wherein microbes in said fermentation fluid are lysed and product is isolated from said broth and said lysed microbes.

18. A method of producing ethanol, said method comprising the steps of:
a) pyrolyzing methane in the presence of a catalyst to split said methane into a hydrogen stream and a solid carbon stream, wherein said pyrolysis does not form a greenhouse gas;
b) partially oxidizing said solid carbon stream in a POX to form a carbon monoxide-rich syngas stream;
c) introducing said syngas stream and an optional hydrogen stream into a bioreactor unit, wherein said bioreactor unit comprises a plurality of bioreactors in parallel;
d) contacting said syngas stream and said optional hydrogen stream with a fermentation fluid comprising microbes and broth in a first subset of said plurality of bioreactors in said bioreactor unit at fermentation conditions;
e) microbially converting said syngas stream to ethanol in said first subset of said plurality of bioreactors;
f) microbially converting said syngas stream to ethanol in a second subset of said plurality of bioreactors while simultaneous removing said ethanol from said first subset of said plurality of bioreactors and recharging said fermentation fluid; and
g) repeating steps c-f and alternating said first and second subsets of bioreactors.

19. The method of claim 18, wherein said syngas stream in said bioreactor is above atmospheric pressure.

20. The method of claim 18, wherein said syngas stream is fed into a bottom end of said bioreactor above atmospheric pressure.
